(19) United States
(12) Patent Application Publication
(10) Pub. No.: US 2003/0186394 A1
(43) Pub. Date: Oct. 2, 2003

(54) Process to prepare and isolate geldanamycin

(76) Inventors: Kevin A. Short, Mattawan, MI (US); John A. Ceglarek, Portage, MI (US); Christopher W. Rysenga, Kalamazoo, MI (US); Omar A. Salman, Kalamazoo, MI (US); Gregory L. Mannes, Kalamazoo, MI (US); Donald L. Gillespie, Kalamazoo, MI (US)

Correspondence Address: Pharmacia & Upjohn Company, Global Intellectual Property, 301 Henrietta Street, Kalamazoo, MI, 49001, US

(21) Appl. No.: 10/367,225

(22) Filed: Feb. 14, 2003

Related U.S. Application Data
(60) Provisional application No. 60/359,484, filed on Feb. 25, 2002.
(60) Provisional application No. 60/381,327, filed on May 17, 2002.

(51) Int. Cl. C12P 17/18

(52) U.S. Cl. 435/119

(57) ABSTRACT

The present invention relates to a process of preparing geldanamycin by culturing Streptomyces hygroscopicus in an aqueous nutrient medium. The aqueous nutrient medium can comprise starch, a starch conversion enzyme, an assimilable source of protein and optionally a further assimilable source of carbon or can comprise an assimilable source of carbon and an assimilable source of protein.

The present invention also relates to a process for the isolation and purification of geldanamycin.

### CROSS-REFERENCE TO RELATED APPLICATIONS

This application claims the benefit of the following provisional applications: U.S. Serial No. 60/359,484, filed Feb. 25, 2002 and U.S. Serial No. 60/381,327, filed May 17, 2002, under 35 USC § 119(e)(i).

### BACKGROUND OF THE INVENTION

1. Field of the Invention

The present invention refers to an improved fermentation and isolation process for preparing geldanamycin using Streptomyces hygroscopicus in an aqueous nutrient medium.

2. Description of the Related Art

Geldanamycin is a natural product of the filamentous bacterium Streptomyces hygroscopicus. It is described in J. Antibiotics 23, 442-447 (1970) and U.S. Pat. No. 3,595,955. Geldanamycin is considered to be the mixture of two unresolved chemical compounds with the formulae C₂₉H₄₀N₂O₉ and C₂₉H₄₂N₂O₉. Recognized as having antiprotozoic activity, this antibiotic was also known to have high activity against human epidermoid carcinoma cells, see U.S. Pat. No. 3,595,955 and J. Antibiotics 24,1182-1188 (1976). Subsequently, geldanamycin antitumor activity has been demonstrated against 60 cell lines, see Cancer Chemother. Pharmacol, 36(4), 305-315 (1995). Furthermore, geldanamycin has been shown to selectively inhibit heat shock protein 90 (hsp90), a molecular chaperone responsible for protein folding and maturation in vivo and which has been found at higher levels in cancerous cells than in normal cells, see J. Biol. Chem., 275(41), 31682-31688 (2000) and Exp. Cell Res., 262(1), 59-68 (2001).

U.S. Pat. No. 3,595,955 discloses the known process to prepare geldanamycin. This process is not amenable to upscale since it only results in product titers of approximately 0.25 g/L. In addition, it employs animal-derived components in the aqueous nutrient medium, which are undesirable due to their cost and the risks of contamination.

It is, therefore, an object of the present invention to provide an improved process of preparing and isolating geldanamycin, which provides increased product titers in the aqueous nutrient medium.

It is a further object of the present invention to provide a novel process of preparing geldanamycin wherein the aqueous nutrient medium is free of animal-derived products.

### SUMMARY OF INVENTION

Disclosed is a process for preparing geldanamycin comprising culturing Streptomyces hygroscopicus in an aqueous nutrient medium containing starch, a starch conversion enzyme and an assimilable source of complex nitrogen while aerating the aqueous nutrient medium at a rate of from about 0.1 to 1.0 volumes air per volume aqueous nutrient medium per minute at least during part of the culturing.

Also disclosed is a process of preparing geldanamycin comprising culturing Streptomyces hygroscopicus in an aqueous nutrient medium comprising an assimilable source of carbon and an assimilable source of complex nitrogen while aerating the aqueous nutrient medium at a rate of from about 0.1 to 1.0 volumes air per volume aqueous nutrient medium per minute at least during part of the culturing.

Further disclosed is a process for the isolation and purification of geldanamycin.

### DETAILED DESCRIPTION OF THE INVENTION

One embodiment the present invention is a process for preparing geldanamycin comprising culturing Streptomyces hygroscopicus in an aqueous nutrient medium containing starch, a starch conversion enzyme and an assimilable source of complex nitrogen while aerating the aqueous nutrient medium at a rate of from about 0.1 to 1.0 volumes air per volume aqueous nutrient medium per minute at least during part of the culturing.

Another embodiment of the present invention is a process for preparing geldanamycin comprising culturing Streptomyces hygroscopicus in an aqueous nutrient medium comprising an assimilable source of carbon and an assimilable source of complex nitrogen while aerating the aqueous nutrient medium at a rate of from about 0.1 to 1.0 volumes air per volume aqueous nutrient medium per minute at least during part of the culturing.

It is preferred that the Streptomyces hygroscopicus microorganism be of the variety geldanus, more preferably Streptomyces hygroscopicus variety geldanus variety nova (the microorganism is available from the Northern Regional Research Laboratory as NRRL 3602).

The aqueous nutrient medium for the invention of the first embodiment, comprises starch, a starch conversion enzyme, and an assimilable source of complex nitrogen. It can also contain a further assimilable source of carbon in addition to the starch. The aqueous nutrient medium for the invention of the second embodiment, does not necessarily contain starch and a starch conversion enzyme, but rather comprises an assimilable source of carbon and an assimilable source of complex nitrogen. The first and second aqueous nutrient medium can further contain nutrient salts, trace elements, antifoam agents and other conventional additives.

Any starch can be used as the assimilable source of carbon in the first aqueous nutrient medium and include for example corn starch, potato starch, white sweet potato starch, grain sorghum starch, tapioca, wheat starch, rice starch, sago, arrowroot and mixtures thereof. Preferably the starch is corn starch, potato starch, wheat starch, rice starch, arrowroot or mixtures thereof. The most preferable starch is corn starch. The starch is typically present at a concentration of from about 10 g/L to about 100 g/L, preferably from about 60 g/L to about 90 g/L, in the aqueous nutrient medium.

In addition to starch the first aqueous nutrient medium can optionally comprise at least one further assimiliable source of carbon such as those, which are conventionally employed in fermentation processes. The further assimilable source of carbon can be, but is not restricted to, glucose, glucose monohydrate, sucrose, mannitol, sorbitol, glycerol, dextrin, fructose, molasses, oatmeal, maltose, lactose or galactose. It can be present in the aqueous nutrient medium in an amount of from about 5 g/L to about 50 g/L, preferably from about 10 g/L to about 30 g/L. Preferably no further assimiliable source of carbon is present.

A further component of the first aqueous nutrient medium is a starch conversion enzyme, which is capable of depolymerizing the starch. The starch conversion enzymes include e.g. endogluconases such as bacterial α-amylase (e.g. validase, Rhozyme, Tenase), fungal α-amylase, amyloglucosidase and various commercially available starch conversion enzymes. The preferred starch conversion enzymes are α-amylases. Typical concentrations of the starch conversion enzyme in the aqueous nutrient medium are from about 10 mg/L to about 100 mg/L.

The second aqueous nutrient medium can contain either starch or any of the assimiliable sources of carbon mentioned above as the assimiliable source of carbon for the fermentation process. The assimiliable source of carbon is preferably contained in the second aqueous nutrient medium in a concentration of from about 10 g/L to about 100 g/L, more preferably from about 60 g/L to about 90 g/L. In the second aqueous nutrient medium a starch conversion enzyme is not necessarily present.

Streptomyces hygroscopicus also requires the presence of an assimilable source of complex nitrogen in the aqueous nutrient medium to be able to produce geldanamycin. The choice of the assimilable source of complex nitrogen is not particularly limited and is known to those skilled in the art. This component can be selected from any of those conventionally employed in corresponding fermentation processes. Examples thereof are, but are not limited to, soybean meal, soybean flour, yeast meal, yeast extract, meat extract, malt extract, cornsteep liquor, peptone, casein, cottonseed oil, molasses, peanut meal, wheat gluten, meat meal, fish meal and mixtures thereof. Of these assimilable sources of complex nitrogen, non-animal-derived products such as soybean meal, soybean flour, corn steep liquor, yeast meal, yeast extract, malt extract and mixtures thereof are preferred. The most preferred protein sources are soybean meal and soybean flour. Generally the assimilable source of complex nitrogen will be present in the aqueous nutrient medium at a concentration of from about 15 g/L to about 150 g/L, preferably from about 20 g/L to about 70 g/L.

Optionally the aqueous nutrient medium can comprise conventional nutrient salts like sodium chloride, magnesium sulfate, potassium chloride, potassium hydrogen phosphate, potassium dihydrogen phosphate, calcium chloride, calcium carbonate, sodium hydrogen phosphate, sodium dihydrogen phosphate, magnesium phosphate, calcium phosphate, as well as inorganic nitrogen sources such as ammonium salts (e.g. ammonium sulfate, ammonium chloride, ammonium nitrate, ammonium acetate, ammonium hydrogen phosphate) and nitrates (e.g. sodium nitrate, potassium nitrate). Combinations of these nutrient salts can also be employed advantageously. Preferred nutrient salts are ammonium salts, calcium carbonate, and mixtures thereof, most preferably ammonium sulfate, calcium carbonate, and mixtures thereof. Trace elements can also be included into the aqueous nutrient medium to further improve the fermentation process. Examples of suitable trace elements are iron, manganese, copper, zinc, nickel, cobalt or other heavy metals as well as mixtures thereof. These are preferably added to the aqueous nutrient medium in the form of their water-soluble salts. The most suitable concentrations for the nutrient salts and trace elements depend on the specific nutrient and elements chosen and can be easily determined by a person skilled in the art. Typical concentrations of the nutrient salts are from about 1 g/L to about 20 g/L, preferably from about 5 g/L to about 12 g/L. The trace elements are usually included in a concentration of from about 5 mg/L to about 1 g/L, preferably from about 10 mg/L to about 0.5 g/L.

In addition to the components mentioned above, the aqueous nutrient medium can contain other conventional additives. Particularly useful are oils, fats and surfactants, which can serve as antifoam agents. Illustrative examples of antifoam agents include, but are not restricted to, polyalkylene glycol (such as ethylene glycol), silicone oil, plant oils (such as soybean oil) and animal oils (such as lard, chicken oil), although the later are generally not preferred. The amount of antifoam agent added to the aqueous nutrient medium depends on the amount of foaming observed, typical amounts, however, are from about 0.1 g/L to about 5 g/L, preferably from about 0.2 g/L to about 2 g/L.

The process for the preparation of the geldanamycin comprises cultivation of Streptomyces hygroscopicus by fermentation in the above-described nutrient media. The geldanamycin, which is thus formed, can subsequently be isolated and purified from the fermentation broth.

First the aqueous nutrient medium is prepared and is filled into a conventional fermentation vessel together with an inoculum of Streptomyces hygroscopicus. Using the aqueous nutrient medium the cultivation can be conducted using any conventional cultivation conditions conducive to satisfactory growth of the microorganism. Usually the fermentation will be conducted under submerged aerobic conditions with agitation. Preferably the agitation should be conducted so that the dissolved oxygen is maintained at 10% to 100% of saturation, more preferably 20% to 40% (as measured by, for example, polarographic dissolution oxygen probe). The temperature during the fermentation will typically be held at from about 18° C. to about 34° C., preferably from about 25° C. to about 30° C. The pH of the aqueous fermentation broth should generally be controlled so that it is in the range of from about 5 to about 8, preferably the pH is in the range of from about 6 to about 7.

It is necessary to aerate the aqueous nutrient medium as is known to those skilled in the art. The aeration is preferably maintained at a rate of from about 0.1 VVM to about 1.0 VVM, preferably from about 0.1 to about 0.5, most preferably from about 0.2 to about 0.3, volumes of air per volume of broth per minute (VVM) at least during part of the culturing. It is also preferred to apply a backpressure at least during part of the culturing, typically during aeration. It is preferred that the aeration be continued during the complete fermentation. In large tanks, however, the hydrostatic pressure should also be taken into account so that the backpressure would be the sum of the hydrostatic pressure and the applied or head pressure. The backpressure should be in the range of from about 3 to about 25 kg/cm², more preferably from about 15 to about 22 kg/cm².

The fermentation is conducted until the desired product titer has been achieved in the aqueous nutrient medium. Using the processes of the invention it is now possible to achieve high geldanamycin titers such as from about 0.5 g/L and higher or about 0.8 g/L and higher or even about 1.0 g/L and higher in the aqueous nutrient medium. The progress of the fermentation and the formation of geldanamycin can be followed by measuring the antibacterial activity as described in U.S. Pat. No. 3,595,955 or by chromatography such as HPLC, see PREPARATION 4. When the desired geldanamycin titer has been reached, the fermentation is stopped and the product can be isolated and purified. The exact length of the fermentation process can vary but will usually be in the range of about 4 to about 10 days, more typically about 6 to about 8 days.

A variety of procedures can be employed to isolate and purify geldanamycin from the fermentation broth, for example, chromatographic adsorption procedures followed by elution with a suitable solvent, column chromatography, partition chromatography, and crystallization from solvents and combinations thereof. U.S. Pat. No. 3,595,955 discloses a method of isolation.

The present invention also includes a novel process for the isolation and purification of geldanamycin. The invention permits isolation and purification in much higher yields than the prior art. The isolation and purification process involves (1) adjusting the pH of the fermentation media to from about 6 to about 7; (2) separation of the biomass from the non-biomass liquid, (3) clarification of the non-biomass liquid of step (2), (4) extraction of the crude geldanamycin, (5) concentration of the extract and (6) crystallization of the crude geldanamycin where steps (4), (5) and (6) are performed with minimum exposure to light. It is preferred that the pH be adjusted to from about 6.4 to about 6.5. The separation of the biomass from the non-biomass liquid can be accomplished by either filtration or centrifugation. It is preferred that the separation be by filtration. When performing the filtering step it is preferred to cool the entire fermentation media from about 0 to about 10°, preferably from about 2 to about 8°. It is preferred to use a filtering aid when performing the filtration. Operable filtering aids are known to those skilled in the art and include diatomaceous earth. It is preferred that the diatomaceous earth be of low and medium porosity and mixtures thereof. It is preferred that the amount of diatomaceous earth used is from about 1 to about 10 Kg/100 L of fermentation media; it is more preferred that the amount of diatomaceous earth used is about 4 Kg/100 L of fermentation media. When performing the clarification step, it is preferred that the clarification is performed using a cellulose based filter media. The extraction is performed with a water immiscible organic solvent. It is preferred that the water immiscible organic solvent is selected from the group consisting of methylene chloride, ethyl acetate, butyl acetate and butanol; it is more preferred that the water immiscible organic solvent is methylene chloride. It is preferred that the concentration step is performed by distillation. It is preferred that the crystallization is from an organic solvent or mixture of organic solvents. It is preferred that the organic solvent is selected from the group consisting of isooctane, heptane and hexane; it is more preferred that the organic solvent is isooctane. It is preferred to perform the crystallization process at a temperature of from about 0 to about 10°. Steps (4), (5) and (6) should be performed with minimum exposure to light. It is preferable to perform steps (1), (2) and (3) with a minimum of light but that is not as critical.

Geldanamycin is know to be a useful pharmaceutical, see J. Antibiotics 23, 442-447 (1970), U.S. Pat. No. 3,595,955, J. Antibiotics 24, 1182-1188 (1976), Cancer Chemother. Pharmacol, 36(4), 305-315 (1995), J. Biol. Chem., 275(41), 31682-31688 (2000) and Exp. Cell Res., 262(1), 59-68 (2001).

Using the process of the present invention, surprisingly it has been found that it is possible to achieve high geldanamycin titers, such as about 0.5 g/L and higher, preferably about 0.8 g/L and higher and even about 1.0 g/L and higher in the aqueous nutrient medium and thus achieve a much higher productivity.

### DEFINITIONS AND CONVENTIONS

The definitions and explanations below are for the terms as used throughout this entire document including both the specification and the claims.

All temperatures are in degrees Celsius.

VVM refers to the volumes of air per volume of broth per minute.

HPLC refers to high pressure liquid chromatography.

psig refers to pounds per square inch gage.

DO refers to dissolved oxygen.

SLM refers to standard liters per minute.

Chromatography (column and flash chromatography) refers to purification/separation of compounds expressed as (support, eluent). It is understood that the appropriate fractions are pooled and concentrated to give the desired compound(s).

Pharmaceutically acceptable refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability.

When the solubility of a solid in a solvent is used the ratio of the solid to the solvent is weight/volume (wt/v).

When the solubility of a solid in a solvent is used the ratio of the solid to the solvent is weight/volume (wt/v).

Backpressure refers to hydrostatic pressure+head pressure.

### EXAMPLES

Without further elaboration, it is believed that one skilled in the art can, using the preceding description, practice the present invention to its fullest extent. The following detailed examples describe how to prepare the various compounds and/or perform the various processes of the invention and are to be construed as merely illustrative, and not limitations of the preceding disclosure in any way whatsoever. Those skilled in the art will promptly recognize appropriate variations from the procedures both as to reactants and as to reaction conditions and techniques.

### PREPARATION 1

### Corn Starch Nutrient Medium

| Ingredient | Concentration | |
|---|---|---|
| Corn Starch | 87.5 | g/L |
| Soy flour | 37.5 | g/L |
| CaCO₃ | 7.5 | g/L |
| FeSO₄ 7H₂O | 112.5 | mg/L |
| Alpha-amylase | 50 | mg/L |

Ingredients are mixed with water, pH adjusted to about ˜7, and volume adjusted to 1-L total volume.

### PREPARATION 2

### Potato Starch Nutrient Medium

| Ingredient | Concentration | |
|---|---|---|
| Soy flour | 37.5 | g/L |
| Potato starch | 87.5 | g/L |
| (NH₄)₂SO₄ | 2.5 | g/L |
| CaCO₃ | 7.5 | g/L |
| CoCl₂.2 H₂O | 10 | mg/L |
| FeSO₄.7 H₂O | 112.5 | mg/L |
| α-amylase | 50 | mg/L |
| Soybean oil | 0.02 | mL/L |

The ingredients are mixed, sufficient water is added to bring the mixture to volume and the pH is adjusted to about 7.

### PREPARATION 3

### Corn Starch Nutrient Medium

A nutrient medium according to the invention which uses corn starch is as follows:

| Ingredient | Concentration | |
|---|---|---|
| Soy flour | 37.5 | g/L |
| Corn starch | 87.5 | g/L |
| Ammonium sulfate | 2.5 | g/L |
| Calcium carbonate | 7.5 | g/L |
| Cobalt chloride dihydrate | 10 | mg/L |
| Ferrous sulfate heptahydrate | 112.5 | mg/L |
| α-amylase | 50 | mg/L |
| Soybean oil | 0.02 | ml/L |

The nutrient medium of PREPARATION 3 was prepared following the general procedure of PREPARATION 2 and making non-critical variations.

### PREPARATION 4

### Assay Procedure for Geldanamycin—Reverse Phase HPLC

The progress of the fermentation process is evaluated by measuring the amount of geldanamycin formed using RP-HPLC. The fermentation broth and the reference sample pure geldanamycin, respectively, are prepared by weighing approximately 0.1 mg thereof into a 20 mL flask, adding 1 mL of acetonitrile and highly purified water (1/1, v/v). The mixture is sonicated until a clear solution is obtained and then injected into the HPLC.

Geldanamycin HPLC Procedure

| Instrumentation Needed | TSP UV-3000 Ultraviolet Detector | | |
|---|---|---|---|
| | TSP P40000 LC Pump | | |
| | AS3000 | | |
| Integration | TSP ChromQuest | | |
| Injection Volumne | 5 μL | | |
| Analysis Time | 40 minutes | | |
| Detector | Data Rate | | 24.0 |
| | Wavelength | | 305 nm |
| | Rise | | 2.0 |
| LC Pump | Flow Rate | | 1.0 ml/minute |
| | Pressure | | High—5500 psi |
| | | | Low-—10 psi |
| Column Heater | Ambient | | |
| Column | Zorbax 300sb-c 18, | | |
| | 4.6 × 150 mm, 5μ | | |
| Reference Standard | | | |
| Reference Sample | | | |
| Mobile Phase | Mobile Phase A: | | |
| | TFA-—0.5 mL | | |
| | ACN-—50.0 mLs | | |
| | Milli-Q H20—949.5 mLs | | |
| | Mobile Phase B: | | |
| | TFA—0.5 mL | | |
| | ACN—950.0 mLs | | |
| | Milli-Q H20—49.5 mLs | | |
| Gradient | Time: | A | B |
| | 0 | 100 | 0 |
| | 25.0 | 0 | 100 |
| | 30.0 | 0 | 100 |
| | 30.1 | 100 | 0 |
| | 40.0 | 100 | 0 |

| | |
|---|---|
| Column Wash | 50/50 Methanol/Milli-Q Water |
| Sample Solvent | Acetonitrile/Milli-Q Water 50/50 |
| Sample Stability | 24 hrs. |
| Calibration (Standard Preparation) | Accurately weigh in |
| | duplicate ˜0.1 mg of the reference |
| | standard into a 20 mL flask. Add |
| | 1 ml of 50% Acetonitrile and Milli- |
| | Q Water, sonicate. Inject. |
| Sample Preparation | XTL—Prepare same as the standard. |

### Example 1

### Fermentation on Shake-Flask Scale

The seed inoculum for shake-flask fermentations is prepared as follows. An ampule culture (100 μL) of Streptomyces hygroscopicus variety geldanus variety nova (NRRL 3602) is inoculated with 100-mL of seed medium (I) in a 500-mL Erlenmeyer flask, sealed with three cotton filters.

Seed medium (I) consists of: 10 g/L glucose monohydrate, 2.5 g/L Yeast extract, and 10 g/L peptone extract. After mixing for 20 minutes, with no pH adjustment, the medium was transferred to a 100-mL Erlenmeyer flask and autoclave sterilized. After inoculation, the seed culture is incubated at 28° C. for three days, on a 2″ (5 cm)-throw shaker at 255 rpm.

The fermentation medium of PREPARATION 3 is inoculated with a 5% seed (5-mL into 100-mL of medium). Fermentation flasks are sealed with three cotton filters, type A400-33. After inoculation, the culture is incubated at 28° C. for two-seven days, on a 2″ (5 cm)-throw shaker at 255 rpm.

The product is isolated, purified and identified as geldanamycin. The amount of geldanamycin produced is 0.5 g/L at 3 days.

### Example 2

### Stirred Tank (Fermentor)—5,000 L tank

A seed medium for use with stirred-tank fermentations is (per L):

| Ingredient | Concentration (g/L) |
|---|---|
| Dextrose | 10 |
| Hy-Soy | 2.5 |
| Yeast Extract | 10 |

For this scale, a primary and secondary seed are required. The medium for the primary and secondary seeds follow the same recipe as EXAMPLE 1, except that the secondary seed also includes 0.04 mL PEG/L (for seed medium I).

Primary flask preparation is mixing of the ingredients, adjusting the pH and dispense 300 mL of the seed medium into a 1 L Erlenmeyer flask, sealed with three cotton filters, type A400-33. The flasks are autoclaved for 45 min. After the flasks are cooled they are inoculated with 1 mL of an ampule (a culture which was previously cultured in primary seed medium (I), mixed with 15% glycerol, and stored at −80° C.). The primary seed is incubated at 28° C. for 3 days on a rotary shaker with a 2″ (5 cm) throw. The entire contents of the primary flask are transferred to a stirred-tank containing 250 L of secondary seed medium. This tank is maintained at 28° C. without pH control, at 7 psi backpressure, 200 SLM, at 250 rpm. After 72 hours of incubation, the entire contents of the seed tank are pushed to the fermentor tank.

The fermentation medium for the 5000 L tank differs from the shake-flask medium in that 0.4 mL of polyalkyleneglycol antifoam is added to help minimize foam. The fermentation is maintained at 28° C. and the pH is controlled between pH 6.25 and 7.75. The back pressure is between 7 and 20 psi with the stirring being between 180-220 rpm. The mixture is aerated between 1000-1500 SLM. The fermentation is allowed to run for 5, 6 or 7 days. The fermentation also received three to four water shots, each of 300 L, on days 3, 4, 5 and 6. At harvest, the entire contents of the tank are pushed to isolation.

| Time (hours) | Amount of Geldanamycin (g/L) |
|---|---|
| 0 | 0 |
| 54 | 0.03 |
| 65 | 0.04 |
| 80 | 0.08 |
| 88 | 0.12 |
| 104 | 0.25 |
| 113 | 0.38 |
| 126 | 0.44 |
| 136 | 0.57 |
| 150 | 0.93 |
| 164 | 1.21 |

### Example 3

### Isolation and Purification of Geldanamycin

About 6,250 L of geldanamycin culture medium is harvested with a titer of 1.250 mg/mL. The culture medium is cooled to 5° (±3°). Then a mixture of diatomaceous earth produced by Eagle-Picher Industries in Reno, Nev. and diatomaceous earth produced by Celite Corporation in Lompac, Calif. filter aids (1/1) is added in an amount of 4 Kg filter aid per 100 L of fermentation medium. The fermentation medium with the filter aid is then filtered on a rotary vacuum filter (36″ diameter). The solids are discarded and the filtrate is collected in a jacketed tank and cooled to 5° (±3°). The pooled filtrate volume is 6,000 L with an activity of 0.32 mg/mL. The discarded solids (spent cake) weighed 1,150.5 Kg with an activity of 3.458 mg/g. The filtrate is subsequently contacted with a cellulose-based filter media (type 10A produced by Cuno Incorporated, Meriden, Conn.) to retain crude geldanamycin crystals. The filter media is extracted with methylene chloride by recycling the solvent through the filter elements for 30-60 minutes. The total volume of the pooled methylene chloride extract is 6,180 L with an activity of 0.171 mg/mL. The rich extract is concentrated by distillation to a volume of 100 L. Then 100 L of isooctane is added over approximately 30 minutes maintaining a pot temperature of 35-40° to crystallize geldanamycin. The crystal slurry is cooled to 5° (±3°) then filtered on a Nutsche type filter. The crystal cake is washed with isooctane (25 L) then dried using 55-65° recycle nitrogen until the loss on dryness (LOD) is <2.5%. The total weight of final dry product was 540.4 grams.

### Example 4

### Isolation and Purification of Geldanamycin

About 6,075 L of geldanamycin culture medium is harvested with titer of 0.872 mg/mL. The culture medium is cooled to 5° (±3°). Then a mixture of diatomaceous earth produced by Eagle-Picher Industries in Reno, Nev. and diatomaceous earth produced by Celite Corporation in Lompac, Calif. filter aids (1/1) is added in an amount of 4 Kg filter aid per 100 L of fermentation medium. The fermentation medium with the filter aid is then filtered on a rotary vacuum filter (36″ diameter). The solids are discarded and the filtrate is collected in a jacketed tank and cooled to 5° (±3°). The fermentation medium is filtered in 3 equal potions. For the first portion, the filtrate volume is 2,500 L with an activity of 0.162 g/L and the spent cake weight is 394.5 Kg with an activity of 2.879 mg/g. For the second portion, the filtrate volume is 2,400 L with an activity of 0.709 g/L and the spent cake weight is 389.5 Kg with an activity of 2.775 mg/g. For the last portion, the medium pH is adjusted to 6.4 before adding filter aid. The filtrate volume from this portion is 2,330 L with an activity of 0.810 g/L and the spent cake weight is 392 Kg with an activity of 0.506 mg/g. The pooled filtrate was subsequently contacted with a cellulose-based filter media (type 10A produced by Cuno Incorporated, Meriden, Conn.) to retain crude geldanamycin crystals. The filter media is extracted with methylene chloride by recycling through the filter elements for 30-60 minutes. The total volume of the pooled methylene chloride extract is 5,850 L with an activity of 0.235 mg/mL. The rich extract is concentrated by distillation to a volume of 115 L. Then 115 L of isooctane is added over approximately 30 minutes maintaining a pot temperature of 35-40° to crystallize geldanamycin. The crystal slurry is cooled to 5° (±3°) then filtered on a Nutsche type filter. The crystal cake is washed with isooctane (2×15 L) then dried using 55-65° recycle nitrogen until the loss on dryness (LOD) is <2.5%. The total weight of final dry product is 1,186.6 grams.

### Example 5

### Isolation and Purification of Geldanamycin

About 6,030 L of geldanamycin culture medium is harvested with titer of 0.758 mg/mL. The culture medium is cooled to 5° (±3°). The culture medium is processed in 3 equal potions. The culture medium pH is adjusted to a 6.4-6.5 range. Then a mixture of diatomaceous earth produced by Eagle-Picher Industries in Reno, Nev. and diatomaceous earth produced by Celite Corporation in Lompac, Calif. filter aids (1/1) is added in an amount of 4 Kg filter aid per 100 L of fermentation media. The fermentation media with the filter aid is then filtered on a rotary vacuum filter (36″ diameter). The solids are discarded and the filtrate is collected in a jacketed tank and cooled to 5° (±3°). The pooled filtrate volume is 7,000 L with an activity of 0.795 mg/mL. The discarded solids (spent cake) weighed 1,146 Kg with an activity of 2.189 mg/g. The filtrate is subsequently contacted with a cellulose-based filter media (type 10A produced by Cuno Incorporated, Meriden, Conn.) to retain crude geldanamycin crystals. The clarified filtrate, which has a volume of 6,565 L and an activity of 0.292, is discarded. The filtered media is extracted with methylene chloride by recycling the solvent through the filter elements for 30-60 minutes. The total volume of the pooled methylene chloride extract is 4,600 L with an activity of 0.44 mg/mL. The rich extract is concentrated by distillation to a volume of 150 L. Then isooctane (150 L) are added over approximately 30 minutes maintaining a pot temperature of 35-40° to crystallize geldanamycin. The crystal slurry is cooled to 50° (±3°) then filtered on a Nutsche type filter. The crystal cake is washed with isooctane (2×15 L) then dried using 55-65° recycle nitrogen until the loss on dryness (LOD) is <2.5%. The total weight of final dry product was 2136.7 grams.

1. A process of preparing geldanamycin comprising culturing Streptomyces hygroscopicus in an aqueous nutrient medium containing starch, a starch conversion enzyme and an assimilable source of complex nitrogen while aerating the aqueous nutrient medium at a rate of from about 0.1 to 1.0 volumes air per volume aqueous nutrient medium per minute at least during part of the culturing.

2. A process according to claim 1, wherein the Streptomyces hygroscopicus is Streptomyces hygroscopicus variety geldanus.

3. A process according to claim 1, wherein the aqueous nutrient medium further comprises an assimilable source of carbon other than starch.

4. A process according to claim 1, wherein the starch is selected from the group consisting of corn starch, potato starch, wheat starch, rice starch, arrowroot and mixtures thereof.

5. A process according to claim 4, wherein the starch is corn starch.

6. A process according to claim 1, wherein the starch conversion enzyme is an α-amylase.

7. A process according to claim 1, wherein the assimilable source of complex nitrogen is selected from the group consisting of soybean meal, soybean flour, cornsteep liquor, yeast meal, yeast extract, malt extract, peptone, casein and mixtures thereof.

8. A process according to claim 1, wherein the aqueous nutrient medium further comprises at least one nutrient salt, at least one trace element or mixtures thereof.

9. A process according to claim 8, wherein the nutrient salts are ammonium sulfate, calcium carbonate or mixtures thereof.

10. A process according to claim 8, wherein the trace elements are iron, cobalt or mixtures thereof.

11. A process according to claim 1, wherein the aqueous nutrient medium comprises:
from about 10 g/L to about 100 g/L of starch,
from about 10 mg/L to about 100 mg/L of starch conversion enzyme,
from about 15 g/L to about 150 g/L of the assimilable source of complex nitrogen,
optionally from about about 5 g/L to about 50 g/L of the further assimilable source of carbon,
optionally from about 0.5 g/L to about 5 g/L of an antifoam agent,
optionally from about 1 g/L to about 20 g/L of nutrient salts and
optionally from about 5 mg/L to about 1 g/L of trace elements.

12. A process according to claim 1, wherein the aqueous nutrient medium comprises:
from about 60 g/L to about 90 g/L of starch,
from about 10 mg/L to about 100 mg/L of starch conversion enzyme,
from about 20 g/L to about 70 g/L of the assimilable source of complex nitrogen,
optionally from about 10 g/L to about 30 g/L of the further assimilable source of carbon,
optionally from about 0.2 g/L to about 2 g/L of an antifoam agent,
optionally from about 5 g/L to about 12 g/L percent by weight of nutrient salts and
optionally from about 10 mg/L to about 0.5 g/L percent by weight of trace elements.

13. A process according to claim 1, wherein the aqueous nutrient medium is aerated at a rate of from about 0.1 to about 0.5 volumes air per volume aqueous nutrient medium per minute or less at least during part of the culturing.

14. A process according to claim 1, wherein a backpressure of from about 3 kg/cm³ to about 25 kg/cm² is applied at least during part of the culturing.

15. A process according to claim 14, wherein a backpressure of from about 15 kg/cm³ to about 22 kg/cm² is applied at least during part of the culturing.

16. A process according to claim 1, wherein the pH of the aqueous nutrient medium is in the range of from about 6 to about 8.

17. A process of preparing geldanamycin comprising culturing Streptomyces hygroscopicus in an aqueous nutrient medium comprising an assimilable source of carbon and an assimilable source of complex nitrogen while aerating the aqueous nutrient medium at a rate of from about 0.1 to 1.0 volumes air per volume aqueous nutrient medium per minute at least during part of the culturing.

18. A process according to claim 17, wherein the Streptomyces hygroscopicus is Streptomyces hygroscopicus variety geldanus.

19. A process according to claim 17, wherein the starch is selected from the group consisting of corn starch, potato starch, wheat starch, rice starch, arrowroot and mixtures thereof.

20. A process according to claim 20, wherein the starch is corn starch.

21. A process according to claim 17, wherein the starch conversion enzyme is an α-amylase.

22. A process according to claim 17, wherein the assimilable source of complex nitrogen. is selected from the group consisting of soybean meal, soybean flour, cornsteep liquor, yeast meal, yeast extract, malt extract, peptone, casein, and mixtures thereof.

23. A process according to claim 17, wherein the aqueous nutrient medium further comprises at least one nutrient salt, at least one trace element or mixtures thereof.

24. A process according to claim 23, wherein the nutrient salts are ammonium sulfate, calcium carbonate or mixtures thereof.

25. A process according to claim 23, wherein the trace elements are iron, cobalt or mixtures thereof.

26. A process according to claim 17, wherein the aqueous nutrient medium comprises:
from about 10 g/L to about 100 g/L of starch,
from about 10 mg/L to about 100 mg/L of starch conversion enzyme,
from about 15 g/L to about 150 g/L of the assimilable source of complex nitrogen,
optionally from about about 5 g/L to about 50 g/L of the further assimilable source of carbon,
optionally from about 0.5 g/L to about 5 g/L of an antifoam agent,
optionally from about 1 g/L to about 20 g/L of nutrient salts and
optionally from about 5 mg/L to about 1 g/L of trace elements.

27. A process according to claim 17, wherein the aqueous nutrient medium comprises:
from about 60 g/L to about 90 g/L of starch,
from about 10 mg/L to about 100 mg/L of starch conversion enzyme,
from about 20 g/L to about 70 g/L of the assimilable source of complex nitrogen,
optionally from about 10 g/L to about 30 g/L of the further assimilable source of carbon,
optionally from about 0.2 g/L to about 2 g/L of an antifoam agent,
optionally from about 5 g/L to about 12 g/L percent by weight of nutrient salts and
optionally from about 10 mg/L to about 0.5 g/L percent by weight of trace elements.

28. A process according to claim 17, wherein the aqueous nutrient medium is aerated at a rate of from about 0.1 to about 0.5 volumes air per volume aqueous nutrient medium per minute or less at least during part of the culturing.

29. A process according to claim 17, wherein a backpressure of from about 3 kg/cm³ to about 25 kg/cm² is applied at least during part of the culturing.

30. A process according to claim 29, wherein a backpressure of from about 15 kg/cm³ to about 22 kg/cm² is applied at least during part of the culturing.

31. A process according to claim 17, wherein the pH of the aqueous nutrient medium is in the range of from about 6 to about 8.

32. A process for isolation and purification of geldanamycin produced by culturing Streptomyces hygroscopicus in an aqueous nutrient medium which comprises:
(1) adjusting the pH of the fermentation media to from about 6 to about 7;
(2) separation of the biomass from the non-biomass liquid;
(3) clarification of the non-biomass liquid of step (2);
(4) extraction of the crude geldanamycin;
(5) concentration of the extract and
(6) crystallization of the crude geldanamycin where steps (4), (5) and (6) are performed with minimum exposure to light.

33. A process for isolation and purification of geldanamycin according to claim 32 where the separation is filtration.

34. A process for isolation and purification of geldanamycin according to claim 32 where the separation is centrifugation.

35. A process for isolation and purification of geldanamycin according to claim 32 where the filtration is performed in a temperature range of from about 0 to about 10°.

36. A process for isolation and purification of geldanamycin according to claim 35 where the filtration is performed in a temperature range of from about 2 to about 8°.

37. A process for isolation and purification of geldanamycin according to claim 35 where the filtration is performed with a filtering aid.

38. A process for isolation and purification of geldanamycin according to claim 37 where the filtering aid is diatomaceous earth.

39. A process for isolation and purification of geldanamycin according to claim 38 where the diatomaceous earth is of low and medium porosity.

40. A process for isolation and purification of geldanamycin according to claim 38 where the amount of diatomaceous earth used is from about 1 to about 10 Kg/100 L of fermentation media.

41. A process for isolation and purification of geldanamycin according to claim 40 where the amount of diatomaceous earth used is about 4 Kg/100 L of fermentation media.

42. A process for isolation and purification of geldanamycin according to claim 32 where the clarification is performed using a cellulose based filter media.

43. A process for isolation and purification of geldanamycin according to claim 32 where the extraction is performed with a water immiscible organic solvent.

44. A process for isolation and purification of geldanamycin according to claim 43 where the water immiscible organic solvent is selected from the group consisting of methylene chloride, ethyl acetate, butyl acetate, butanol, methyl ethyl ketone.

45. A process for isolation and purification of geldanamycin according to claim 44 where the water immiscible organic solvent is methylene chloride.

46. A process for isolation and purification of geldanamycin according to claim 32 where the concentration is performed by distillation.

47. A process for isolation and purification of geldanamycin according to claim 32 where the crystallization is from an organic solvent or mixture of organic solvents.

48. A process for isolation and purification of geldanamycin according to claim 47 where the organic solvent is selected from the group consisting of isooctane, heptane and hexane.

49. A process for isolation and purification of geldanamycin according to claim 48 where the organic solvent is isooctane.

50. A process for isolation and purification of geldanamycin according to claim 47 where the crystallization process is performed at a temperature of from about 0 to about 10°.

51. A process for isolation and purification of geldanamycin according to claim 32 where the pH is adjusted to from about 6.4 to about 6.5.
